(19) United States
(12) Patent Application Publication
(10) Pub. No.: US 2024/0238110 A1
(43) Pub. Date: Jul. 18, 2024

(54) Vascular Prosthesis With Drawn Filled Tubes

(71) Applicant: MicroVention, Inc., Aliso Viejo, CA (US)

(72) Inventors: Dieter Rutzen, Aliso Viejo, CA (US); Ponaka Pung, Aliso Viejo, CA (US); Shameem Golshan, Aliso Viejo, CA (US); Helen Nguyen, Aliso Viejo, CA (US); Oanh Nguyen, Aliso Viejo, CA (US); Britney Ngo, Aliso Viejo, CA (US); Minh Nguyen, Aliso Viejo, CA (US); Marcus Kraus, Aliso Viejo, CA (US); Nazanin Nourani, Aliso Viejo, CA (US)

(73) Assignee: MicroVention, Inc., Aliso Viejo, CA (US)

(21) Appl. No.: 18/561,684

(22) PCT Filed: May 18, 2022
(86) PCT No.: PCT/US2022/072412

§ 371 Date: Nov. 16, 2023

Related U.S. Application Data
(60) Provisional application No. 63/189,993, filed on May 18, 2021.

(51) Int. Cl. A61F 2/852; A61F 2/82; A61F 2/848; A61F 2/90; A61L 31/02; A61L 31/18

(52) U.S. Cl. CPC A61F 2/852; A61F 2/848; A61F 2/90; A61L 31/022; A61L 31/18; A61F 2002/828

(57) ABSTRACT

A vascular implant may comprise at least a first layer having one or more drawn fill tube wires (DFT wires) and at least a second layer having one or more non-DFT wires. The first layer may be braided from only a single DFT wire, and the second layer may be braided from a plurality of non-DFT wire. A vascular implant may also include a connecting wire composed of a shape memory alloy and that is shape set prior to connection to one or more implant layers composed of DFT wires.

### RELATED APPLICATIONS

This application claims priority to U.S. Provisional Application Ser. No. 63/189,993 filed May 18, 2021 entitled Radiopaque Vascular Prosthesis, which is hereby incorporated herein by reference in its entirety.

### BACKGROUND OF THE INVENTION

Vascular prostheses such as stents and stent-grafts are used for a variety of reasons in the vasculature. A non-exhaustive list includes propping open diseased or occluded vessels to promote blood flow, flow diversion involving diverting flow away from target areas such as aneurysms, and retaining material (e.g., embolic material) within a treatment site to promote localized occlusion within a region.

Visualization remains important for the delivery of vascular prostheses so that a surgeon can confirm proper placement of the device in the vasculature. Typically, radiography is used for such visualization, which is an imaging technique using X-rays, gamma rays, or similar ionizing radiation and non-ionizing radiation to view the form of an object within a patient. Specific types of radiography include static X-rays, CT scans, and fluoroscopy. For structures to be displayed by most radiography, it must be relatively radiopaque. For that reason, one or more radiopaque components are often included on most implantable vascular prostheses.

### SUMMARY OF THE INVENTION

The present invention generally relates to a vascular prosthesis composed of one or more drawn filled tube wires (DFT wires).

One aspect of the present invention is generally directed to an implant having at least one braided layer composed of one or more DFT wires and at least one braided inner layer composed of one or more wires (e.g., DFT wires or non-DFT wires).

The implant may have a variety of different layer configurations of braided DFT wire layers and non-DFT wire layers. For example, the implant may have two braided layers in which the outer layer is composed of DFT wires and the inner layer is composed of non-DFT wires, or in which the outer layer is composed of non-DFT wires and the inner layer is composed of DFT wires. In another example, the implant may have three braided layers in which the layer composed of DFT wires is the outer layer, middle layer, or inner layer, and the two remaining layers composed of non-DFT wires are the remaining layers. In another example, the implant may have three braided layers in which a layer composed of non-DFT wires is the outer layer, the middle layer, or inner layer, and the two remaining layers composed of DFT wires are the remaining layers. In yet another example, the implant may be composed of four or more layers with layers alternating between braided DFT wire layers and non-DFT wire layers (e.g., the DFT wire layer may compose the outermost layer or the non-DFT wire layer may compose the non-DFT wire layer).

In addition to having different combinations of DFT wire and non-DFT wire layers, the implant layers may have different lengths relative to each other. For example, a braided DFT wire layer may extend beyond the proximal and/or distal end of the non-DFT wire layer(s) or a non-DFT wire layer may extend beyond the proximal and/or distal end of the DFT wire layer(s).

Another aspect of the present invention is generally directed to a vascular implant (e.g., a stent or graft) having one or more connecting wires that are non-DFT wires, preferably composed of a shape memory material, and that may be pre-shaped (e.g., heat set) to a desired secondary shape and then connected and/or braided into one or more braided layers of the implant. By pre-shaping the connecting wire to a desired secondary shape, the connecting wire may provide additional force to an implant to achieve its desired expanded configuration size and may help maintain the expanded configuration, particularly in tortuous vessels. As discussed herein, DFT wires may be relatively flexible as compared to non-DFT wires, especially after being heat set. Hence, a connecting wire that is pre-shaped to a desired expanded size may help force the other layers of the implant, including those with DFT wires, to achieve or maintain a desired radial size and potentially better anchor within a patient's vasculature.

The implant may include a single layer, two layers, three layers, or more than three layers. The implant may also include at least one layer braided from DFT wire, and optionally a plurality of layers (e.g., 2 or 3) that are braided from DFT wire. As in previously discussed embodiments, the remaining layers may be composed of non-DFT wires.

The pre-shaped connecting wire may form a helical shape or may be one or more circular shapes. A single connecting wire may be used with an implant or a plurality of connecting wires may be used with an implant. The one or plurality of connecting wires may each extend along the entire length of the stent (or most of the length of the stent) or the one or plurality of connecting wires may extend along only a fraction of the length of the implant (e.g., a quarter, third, half, or three-quarters of the length of the implant).

A plurality of separate connecting wires may be used in a non-overlapping configuration. For example, one connecting wire may extend along a first half of an implant and a second connecting wire may extend along a second half of an implant. Similar configurations may be possible for 3, 4, 5, 6, or more connecting wires. Alternately, a plurality of connecting wires can be arranged so that only portions of each connecting wire overlap in their position along the implant length.

The connecting wires may be connected to one or a plurality of implant layers by interweaving the one or more connecting wires through each of the implant layers and/or by connecting the one or more connecting wires via a connection mechanism to wire locations on the stent, such as welding, rings, wire coils, wire ties, coiling the ends of the connecting wires, or similar techniques. The connecting wires may be used in only a single layer stent embodiment to help open the implant, a two-layer stent embodiment to help connect the layers, or a three or more layer stent embodiment to help connect at least two of the implant layers generate additional radial opening force.

In one example, the connecting wire is any shape memory alloy, such as Nitinol. The connecting wire may be pre-shaped by winding on a mandrel to form a desired size and pattern, and then heat set to establish the desired secondary shape of the connecting wire. The connecting wire may then be connected (e.g., interwoven or fixed to) the one or plurality of layers of an implant. The connecting wire may have a similar shape as one or more portions of the wire of a layer of an implant (i.e., it may closely follow the shape of a portion of one of the implant's wires) or it may have a different pattern/shape than the wire portions of an implant.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which:

FIG. 1 illustrates a cross section of a DFT wire used in a DFT stent, according to one embodiment.

FIG. 2A illustrates a side view of a dual layer DFT stent, according to one embodiment.

FIG. 2B illustrates a photograph side view of the dual layer DFT stent of FIG. 2A, according to one embodiment.

FIG. 3A illustrates an end view of the dual layer DFT stent of FIG. 2A, according to one embodiment.

FIG. 3B illustrates a photograph end view of the dual layer DFT stent of FIG. 2A, according to one embodiment.

FIG. 3C illustrates a magnified view of end loops of a dual layer DFT stent, according to one embodiment.

FIG. 4 illustrates a magnified view of the dual layer DFT stent of FIG. 2A, according to one embodiment.

FIG. 5 illustrates a magnified view of the dual layer DFT stent of FIG. 2A, according to one embodiment.

FIG. 6 illustrates a magnified view of the dual layer DFT stent of FIG. 2A, according to one embodiment.

FIG. 7 illustrates a magnified view of the dual layer DFT stent of FIG. 2A, according to one embodiment.

FIG. 8 illustrates an end view of another embodiment of a stent, according to one embodiment.

FIG. 9 illustrates an end view of another embodiment of a stent, according to one embodiment.

FIG. 10 illustrates a connecting wire on a mandrel, according to one embodiment.

FIG. 11 illustrates a connecting wire connected to a stent wire, according to one embodiment.

FIG. 12 illustrates a side view of a single layer stent, according to one embodiment.

FIG. 13 illustrates an end view of a DFT stent end loop configuration, according to one embodiment.

FIG. 14 illustrates an end view of a DFT stent end loop configuration, according to one embodiment.

FIG. 15 illustrates a planar view of a DFT stent end loop configuration, according to one embodiment.

FIG. 16 illustrates a side view of a stent with a reinforcing member, according to one embodiment.

FIG. 17 illustrates an enlarged view of a stent with a reinforcing member, according to one embodiment.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements. While different embodiments are described, features of each embodiment can be used interchangeably with other described embodiments. In other words, any of the features of each of the embodiments can be mixed and matched with each other, and embodiments should not necessarily be rigidly interpreted to only include the features shown or described.

While the embodiments described in this specification may are generally referred to as stents, the teachings herein are applicable to a wide range of different vascular devices such as grafts, valves, anchoring mechanism, or any other vascular medical device that may include at least one braided portion. Hence, the term stent should be understood to be inclusive of all of these devices.

The present specification also describes several different features of stents, such as different wire materials of different layers, different layer arrangements, different layer lengths, different connections between layers, and other features. Any of these aspects can be used and interchanged with each other. Hence, while every permutation of feature is not specifically described, such combinations are specifically contemplated as being part of the present invention and supported by the specification.

This specification also refers to the use of drawn filled tube wires (DFT wires) and non-DFT wires. The non-DFT wires 16 can be composed of any material typically used for medical devices, including shape memory alloys (e.g., Nitinol), stainless steel, cobalt-chromium, polymers, or other materials. Shape memory alloys, and especially Nitinol, may be preferable in some embodiments. These non-DFT wires 16 are generally composed of a single material through its cross section, though coatings and similar features are also possible.

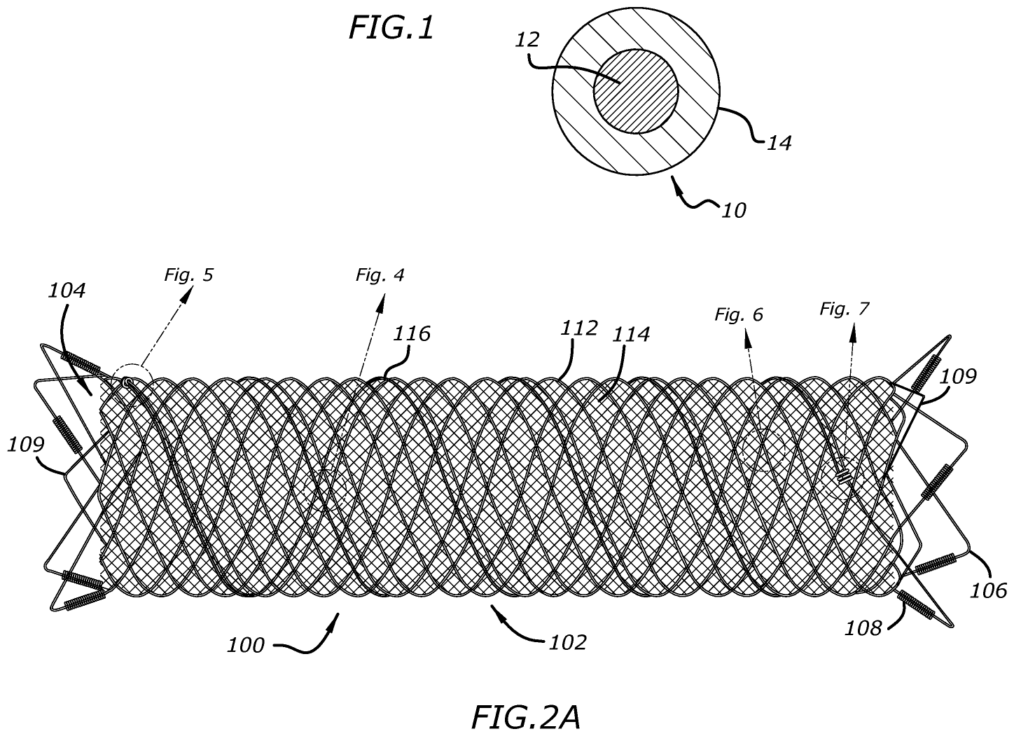

The DFT wires 10 can be composed of a variety of different materials with different cross-sectional thicknesses. For example, FIG. 1 illustrates a cross section of a DFT wire 10 having an inner core 12 composed of a first material and an outer jacket 14 composed of a second material. In another example, the outer jacket 14 may alternately be composed of multiple layers of different material (e.g., two or more layers over inner core 12). Either of the inner core 12 and the outer jacket 14 can be composed of radiopaque materials (such as platinum, gold, tantalum, palladium, or similar known radiopaque materials). Either of the inner core 12 and the outer jacket 14 can be composed of non-radiopaque materials (i.e., materials with a relatively low or no radiopaque properties). Such non-radiopaque materials may include, e.g., stainless steel, cobalt-chromium, or shape memory alloys such as Nitinol. In one example, the inner core 12 may be composed of radiopaque material(s) and the outer jacket 14 may be composed of non-radiopaque materials. In another example, the inner core 12 may be composed of non-radiopaque materials and the outer jacket 14 may be composed of non-radiopaque materials.

In one example, the inner core 12 may be composed of a radiopaque material and the outer jacket 14 may be composed of a shape memory alloy such as Nitinol. The radiopaque material promotes visualization of the DFT wire 10, while the outer jacket 14 allows for good pliability and the ability to have a memorized shape (e.g., via being heat-set). In another example, inner core 12 may be composed of platinum or tantalum, while the outer jacket 14 may be composed of Nitinol-1 or Nitinol-2.

The inner core 12 may have a cross sectional shape that is circular, elliptical, or ovular, though a variety of other shapes can be used, such as rectangular, triangular, or the like. The outer jacket 14 may be tubular in shape with an inner diameter that closely matches an outer diameter of the inner core 12. Put differently, the outer jacket 14 may include an internal lumen through which the inner core 12 extends.

Additionally, DFT wires 10 may sometimes exhibit a higher degree of bendability and reduced stiffness than a purely metallic shape memory wire once heat treatment/heat-setting occurs. This may be generally unexpected since inclusion of a radiopaque material in the inner core 12 (depending on which particular material is used) can generally be stiffer in comparison to the metallic shape memory outer jacket 14. However, the inclusion of two separate materials in creating a single wire can alter the material characteristics of the combined wire shape. Due to these characteristics, when DFT wires 10 are used in a stent, design aspects of the stent may need to compensate for this increased flexibility, especially to promote proper deployment and proper apposition of the DFT stent at the treatment site to prevent stent migration. The embodiments presented herein address these and other issues to create a usable DFT stent.

The outer diameter of the DFT wire 10 may have a wide range of diameters, depending on its use within a stent. For example, the DFT wire 10 may have a diameter within an inclusive range of about 0.001 inch to 0.004 inch, or about 0.0025 inch to about 0.003 inch. The inner core 12 and outer jacket 14 of the DFT wire 10 may be composed of different percentages of the cross section of the DFT wire 10 based on cross-sectional width or diameter. For example, the inner core 12 may be within an inclusive range of 5% to 30% of the cross-sectional width or diameter of the DFT wire 10 with the remaining percentage being the outer jacket 14 (i.e., 95% to 70%). In a more specific example, the ration may be 10% inner core 12 cross sectional width or diameter and 90% outer jacket 14 cross sectional width or diameter.

In some examples, the total cross-sectional width or diameter of the DFT wire 10 is within an inclusive range of about 0.0018 inch to about 0.0022 inch. In some examples, the inner core 12 (e.g., composed of a radiopaque material) has a width or diameter within an inclusive range of about 0.0005 inch to about 0.001 inch, or an inclusive range of about 0.0008 inch to about 0.0009 inch.

Any of the wires 10, 16 used in a stent may be functionalized, for example with poly (MEA-co-APMA).

One aspect of the present invention is generally directed to a stent having at least one braided layer composed of one or more drawn filled tube wires (DFT wires) and at least one braided inner layer composed of one or more wires (e.g., DFT wires or non-DFT wires).

The stent may have a variety of different layer configurations of braided DFT wire layers and non-DFT wire layers. For example, the stent may have two braided layers in which the outer layer is composed of one or more DFT wires, and the inner layer is composed of non-DFT wires, or that the outer layer is composed of non-DFT wires and the inner layer is composed of DFT wires. In another example, the stent may have three braided layers in which the layer composed of DFT wires is the outer layer, middle layer, or inner layer, and the two remaining layers composed of non-DFT wires are the remaining layers. In another example, the stent may have three braided layers in which a layer composed of non-DFT wires is the outer layer, the middle layer, or inner layer, and the two remaining layers composed of DFT wires are the remaining layers. In yet another example, the stent may be composed of four or more layers with layers alternating between braided DFT wire layers and non-DFT wire layers (e.g., the DFT wire layer may composed the outermost layer, or the non-DFT wire layer may compose the non-DFT wire layer).

In addition to having different combinations of DFT wire and non-DFT wire layers, the stent layers may have different lengths relative to each other. For example, a braided DFT wire layer may extend beyond the proximal and/or distal end of the non-DFT wire layer(s) or a non-DFT wire layer may extend beyond the proximal and/or distal end of the DFT wire layer(s).

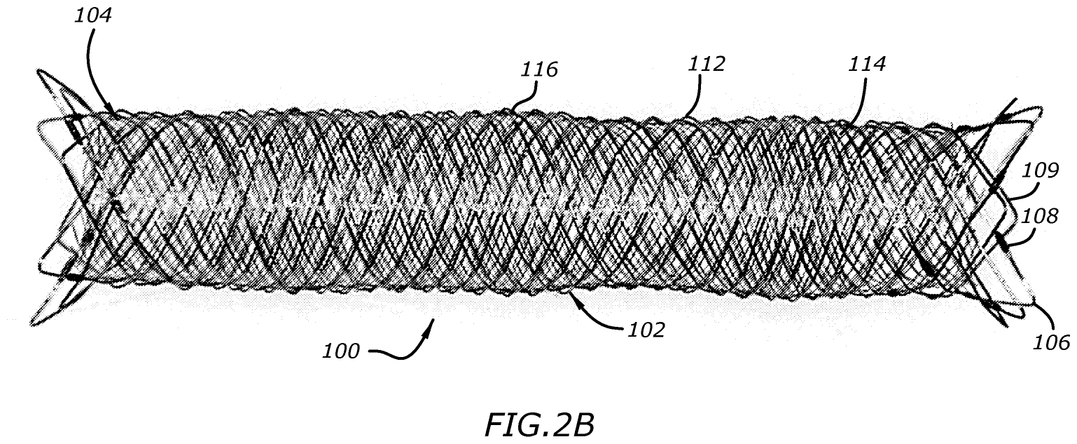

FIGS. 2A-7 illustrate one specific embodiment of a stent 100 having at least one layer composed of one or more DFT wires 10 and at least one layer composed of one or more non-DFT wires 16. More specifically, the stent 100 may include a braided outer layer 102 forming a tubular shape composed of one or more outer wires 112 that are DFT wires 10 and a braided inner layer 104 forming a tubular shape within the outer layer 102 that is composed of one or more inner wires 114 that are non-DFT wires 16. The DFT wires 10 may have any of the previously discussed characteristics, but preferably have an inner core 12 composed of a radiopaque material and an outer jacket 14 composed of a shape memory alloy (e.g., Nitinol).

The use of DFT wires 10, particularly with radiopaque materials comprising the inner core 12, can provide several advantages. First, the DFT wires 10 of the outer layer 102 may be radiopaque and therefore show up on radiography visualization. Unlike the use of relatively small radiopaque markers, the entire outer layer 102 may be visualized which may allow for a physician to better view and place the stent 100. Since radiopaque markers may not be necessary, the lack of such markers may further decrease the profile or thickness of a stent.

Additionally, when a radiopaque material is used in the DFT wire(s) 10, it may have a relatively higher flexibility or bendability than many non-DFT wires 16 composed of shape memory alloys (e.g., Nitinol) because of the properties of the material used in the DFT wire 10 and/or after being heat set to impart a shape to the wire. Hence, a stent layer composed of one or more DFT wires 10 may sometimes better conform to a shape of a tortuous anatomical site within a patient.

The stent 100 may also include several other features discussed further below that may be helpful in connection with DFT wire 10 and non-DFT wire 16 stent layers, though they are not necessarily required. Note, Areas labeled FIG. 4-7 in FIG. 2A correspond to magnified views in FIGS. 4-7, respectively, and FIG. 2B illustrates a photograph view of FIG. 2A.

In one example, the stent 100 may include a tubular shaped outer layer 102 and a tubular shaped inner layer 104 that is attached to the outer layer 102. The outer layer 102 can be configured to anchor the stent 100 within a patient while the inner layer 104 may be less porous than the outer layer 102 so as to help divert or prevent blood flow from passing through.

Both the inner layer 104 and the outer layer 102 may be braided in a helical braiding pattern so that the wires have the same or similar braid angles. This may allow both layers 102 and 104 to increase and decrease in length at the same or similar rate when the stent 100 radially expands or contracts between its radially compressed configuration and its radially expanded configuration. Alternately, the layers 102, 104 may have different braid patterns and/or braid angles.

The outer layer 102 may have a larger pore size or a lower pick per inch (PPI) than the inner layer 104. In one example, the pores may be sized within an inclusive range of about 0.3 mm to about 0.5 mm when the stent 100 is in its expanded configuration. In another example, the braided tubular portion of the outer layer 102 may have a pick per inch within an inclusive range of about 60 PPI to about 85 PPI, and more specifically about 72. However, in some embodiments, the pore/cell sizes and/or the pick per inch of the respective layers 102, 104 may be the same or similar.

The outer wire 112 of the outer layer 102 may have a larger diameter than the inner wire 114 of the inner layer 104. For example, the outer wire 112 of the outer layer 102 may have a diameter within an inclusive range of about 0.001 inch to 0.004 inch, or about 0.0025 inch to about 0.003 inch. In one example, the outer wire 112 of the outer layer 102 may have a diameter of about 0.0016 inch throughout its braided tubular portion and a diameter of about 0.0020 inch along portions of the outer wire 112 forming its end loops 106, 109.

The outer layer 102 may be braided from a single outer wire 112 (e.g., a DFT wire 10) into its tubular shape. Alternatively, the outer layer 102 may be braided from a plurality of outer wires 112 (e.g., DFT wires 10) into its tubular shape. Again, in other embodiments, these outer layer configurations may use non-DFT wire 16 instead. Example diameter sizes for the outer layer 102 in its expanded configuration include 2.5 mm-3.0 mm, 3.5 mm-4.5 mm, 4.5 mm-5.0 mm, 5.0 mm-5.5 mm, 5.5 mm-6.0 mm, and 6.0 mm-8.0 mm with various lengths.

The inner layer 104 may be braided from a single inner wire 114 (e.g., a non-DFT wire 16) into its tubular shape. Alternatively, the inner layer 104 may be braided from a plurality of inner wires 114 (e.g., non-DFT wires 16) into its tubular shape. Again, in other embodiments, these inner layer configurations may use DFT wire 10 instead. The inner layer 104 may form a braided, tubular shape that may be sized to expand to an outer diameter equal to or almost equal to the inner diameter of the outer layer 102. The inner layer 104 can be composed of one or more inner wires 114 (e.g., 20, 24, 36 wires) braided with each other to form its tubular shape. In either wire example, the wire diameter may be about 0.00085 inch and be braided to form about 165 picks per inch in an example embodiment.

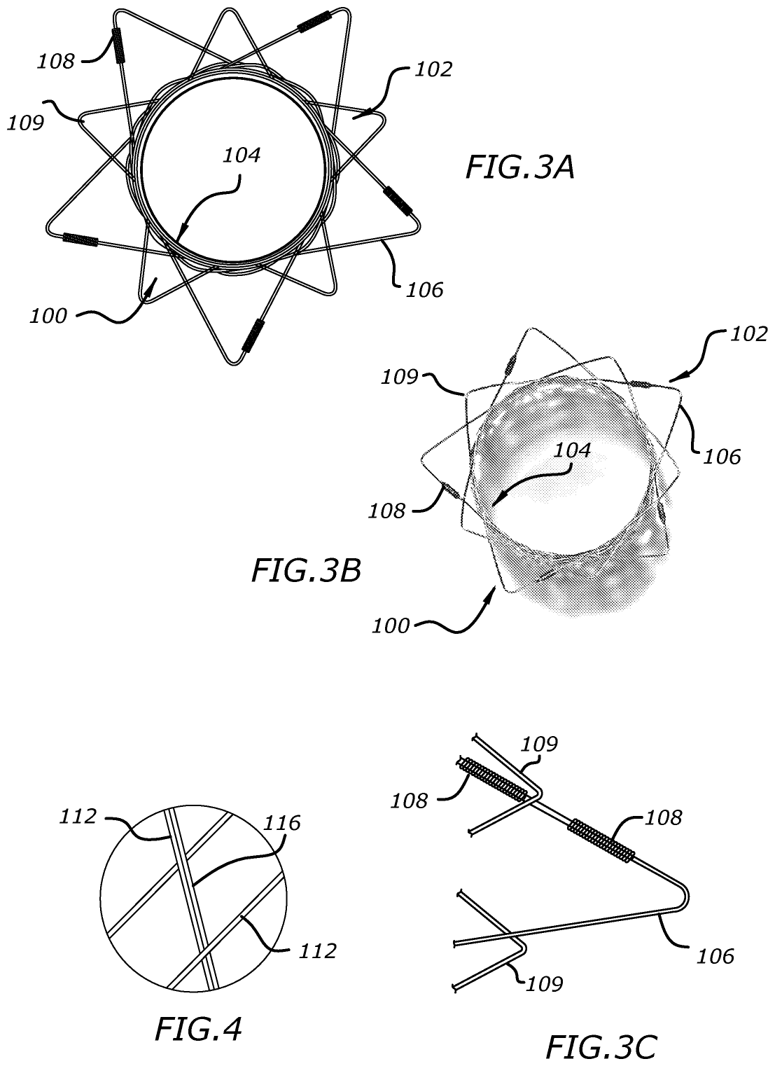

The outer layer 102 may form a braided, tubular shape with a plurality of end loops that can be the same size or different sizes. The loops can be located on the proximal end, the distal end, or both ends. Each end of the braided tubular portion may have, for example, 3, 4, 5, 6, 7, 8, 9, 10, 11, 12, or more loops. The loops may have larger and smaller sizes, such as larger loops 106 and smaller loops 109 in FIG. 3A. These larger and smaller loops 106, 109 may form an alternating pattern as seen in FIG. 3A, where five larger and five smaller loops are interposed with each other. Note, FIG. 3B illustrates a photograph view of FIG. 3A.

As seen in FIG. 6, the ends of the outer wires 112 may be located near each other, such as about three-quarters along the stent's 100 length. The wire ends may be positioned to overlap each other and then one or more (e.g., four) laser welds 112A may be created to connect the portions of the wire 112, thereby preventing the ends or edges of the wire 112 from being easily exposed in a location that may cause damage to the patient. Alternately, other connection mechanisms are possible for these wire ends, such as being tied together or positioned under a separate coil or band.

When DFT wires 10 are used that include a radiopaque inner core 12, additional radiopaque markers may not be necessary. However, depending on which layers incorporate the radiopaque DFT wires 10, radiopaque markers may be helpful, particularly on the ends of the stent 100 to help identify where the stent 100 terminates. In one example such as shown in FIG. 3C, the end loops 106 of the outer layer 102 may also include one or more wire coils 108 (alternately sleeves, tubes, or similar shapes) wrapped around a portion of the outer wire 112 of an end loop 106. The wire coils 108 may be composed of a radiopaque material such as tantalum to help indicate the ends of the stent 100 under imaging and provide additional anchoring force. However, non-radiopaque material may alternately be used. In one example, each end may include four tantalum wire coils 108, the coils 108 may be each positioned near the apex or near the furthermost end of each loop 106, and/or each coil 108 may be formed from tantalum wire having a diameter of about 0.0015 inch. Alternately, the wire coils 108 can be composed of a non-radiopaque material such as Nitinol and may be provided only for anchoring purposes.

The coils 108 can be placed at any location on the loops 106 (or optionally the loops 109). As seen in FIG. 3C, the coil 108 can be positioned closer to the terminal end of a loop or closer to the body of the stent. The embodiment of FIGS. 2A-7 illustrate only the larger loops 106 having coils 108 positioned relatively closer to the terminal ends of the loops 106. In another embodiment, the loops 106 may be positioned closer to the body of the stent (i.e., the left-most coil 108 as shown in FIG. 3C). In this example, the coil 108 is further positioned within the smaller loop 109 such that it remains within the smaller loop 109 when the stent is both in its radially compressed configuration and radially expanded configuration. Since the loops 106 and 109 may move somewhat relative to each other during radial expansion, this positioning may help prevent the coil 108 from moving against the wires of the smaller loop 109, allowing for a smoother opening movement of the stent 100. In another example, a loop may include two coils 108 at both inner and outer locations, as seen in FIG. 3C.

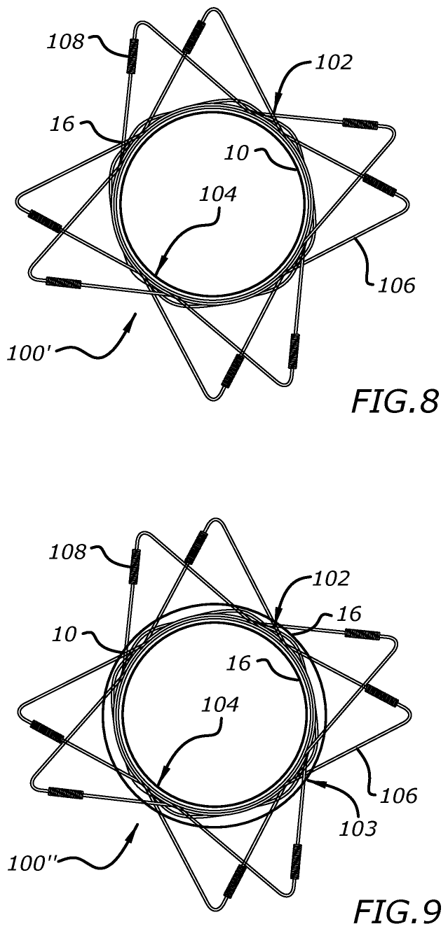

FIG. 8 illustrates another embodiment of a stent 100′ that is generally similar to the previously described stent 100 embodiment but in which the outer layer 102 may be composed of non-DFT wires 16 and the inner layer 104 is composed of DFT wires 10.

FIG. 9 illustrates another embodiment of stent 100″ that is generally similar to the previously described stent 100 embodiment, but which includes a third outer layer 103. Both the third outer layer 103 and the inner layer 104 may be composed non-DFT wires 16 and the middle layer 102 may be composed of DFT wires 10. Alternately, any combination of DFT wires 10 and optionally non-DFT wires 16 can be used for each layer. For example, all layers may be composed of DFT wires 10, only one of the layers 102, 103, 104 can be composed of DFT wires 10 with the remaining being non-DFT wires 16, or two of the layers 102, 103, 104 can be composed of DFT wires 10 with the remaining layer being non-DFT wires 16.

Another aspect of the present invention is generally directed to a vascular device (e.g., a stent or graft) having one or more connecting wires 116 that may be non-DFT wires 16, preferably composed of a shape memory material, and that may be pre-shaped (e.g., heat set) to a desired secondary shape and then connected and/or braided into to one or more braided layers of a stent. By pre-shaping the connecting wire 116 to a desired secondary shape, the connecting wire 116 may provide additional force to a stent to achieve and/or maintain its desired open configuration size. As previously discussed, DFT wires 10 may be relatively flexible as compared to non-DFT wires 16, depending on their material composition. Hence, a connecting wire 116 that is pre-shaped to a desired expanded size may help force the other layers of the stent, including those with DFT wires 10 to achieve a desired radial size and potentially better anchor within a patient's vasculature.

The stent may include a single layer, two layers, three layers, or more than three layers. The stent may also include at least one layer braided from DFT wire 10, and optionally a plurality of layers (e.g., 2 or 3) that are braided from DFT wire 10. As in previously discussed embodiments, the remaining layers may be composed of non-DFT wires 16.

The pre-shaped connecting wire 116 may form a helical shape or may be one or more circular shapes. A single connecting wire 116 may be used with a stent or a plurality of connecting wires 116 may be used with a stent. The one or plurality of connecting wires 116 may each extend along the entire length of the stent (or most of the length of the stent) or the one or plurality of connecting wires 116 may extend along only a fraction of the length of the stent (e.g., a quarter, third, half, or three-quarters of the length of the stent).

A plurality of separate connecting wires 116 may be used in a non-overlapping configuration. For example, one connecting wire 116 may extend along a first half of a stent and a second connecting wire 116 may extend along a second half of a stent. Similar configurations may be possible for 3, 4, 5, 6, or more connecting wires 116. Alternately, a plurality of connecting wires 116 can be arranged so that only portions of each connecting wire overlap in their position along the stent length.

The connecting wires 116 may be connected to one or a plurality of stent layers by interweaving the one or more connecting wires 116 through each of the stent layers and/or by connecting the one or more connecting wires 116 via a connection mechanism to wire locations on the stent, such as welding, rings, wire coils, wire ties, coiling the ends of the connecting wires 116, or similar techniques. The connecting wires 116 may be used in only a single layer stent embodiment to help open the stent, a two-layer stent embodiment to help connect the layers, or a three or more layer stent embodiment to help connect at least two of the stent layers generate additional radial opening force.

In one example, the connecting wire 116 may be any shape memory material, such as Nitinol. The connecting wire 116 may be pre-shaped by winding on a mandrel to form a desired size, shape, and pattern, and then heat set to establish the desired secondary shape of the connecting wire 116. The connecting wire 116 may then be connected (e.g., interwoven or fixed to) the one or plurality of layers of a stent. The connecting wire 116 may have a similar shape as one or more portions of the wire of a layer of a stent (i.e., it may closely follow the shape of a portion of one of the stent's wires) or it may have a different pattern/shape than the wire portions of a stent.

While the connecting wire 116 is described as being pre-shaped, it may alternately be woven with one or more layers of a stent and heat set with the other layers of the stent.

Returning to the example embodiment of FIGS. 2A-7, the use of one or more connecting wires 116 is illustrated. In the final form of the stent 100, the one or plurality of connecting wires 116 may be positioned adjacent to a portion of outer wire 112 in a helical pattern such that is has a similar braid axis and braid angle as outer wire 112. Again, a single connecting wire 116 can be used or a plurality of connecting wires 112 in different arrangements/positions can be used.

The connecting wire 116 may be interwoven with both wires 112 and 114 of both layers 102 and 104 (e.g., an over-under pattern through both), so that both layers are relatively closely positioned adjacent to each other. The similar braid angle allows the wires 112, 114, and 116 of the stent to move in relative unison to foreshorten/lengthen during radial expansion and contraction. In one specific example, two helically woven connecting wires 116 are included, though 1, 3, 4, 5, 6, or more connecting wires 116 can also be included.

By pre shaping and/or heat setting the connecting wire(s) 116, the diameter and pitch of the helical may be defined to perform similarly to the wires 112, 114 forming the inner and outer layers 102, 104. This can be clinically advantageous, since having all components radially expand and foreshorten similarly allows the stent 100 to open and conform better to tortuous anatomies, providing better wall apposition. Thus, stent opening and stability issues may be reduced or eliminated.

In the present embodiment of stent 100, the connecting wire 116 may be comprised of one or more Nitinol helical wires (i.e., a helical, heat set, secondary shape). The use of such Nitinol helical wires or coils instead of non-shape memory wires may allow the stent 100 to open up to a greater size (e.g., to a greater OD such as more than 5 mm) since the pre-shaped and heat set configuration of the connecting wires 116 may generate additional outward radial force, depending on the pre-shaped size of the connecting wires 116 and the otherwise expanded size of the other layers of the stent 100.

The connecting wire 116 may also or alternatively be electropolished prior to use connecting the inner and outer layers 102, 104 in some embodiments. However, it should be appreciated that, in some embodiments, the connecting wire 116 may not be electropolished prior to use connecting the inner and outer layers 102, 104.

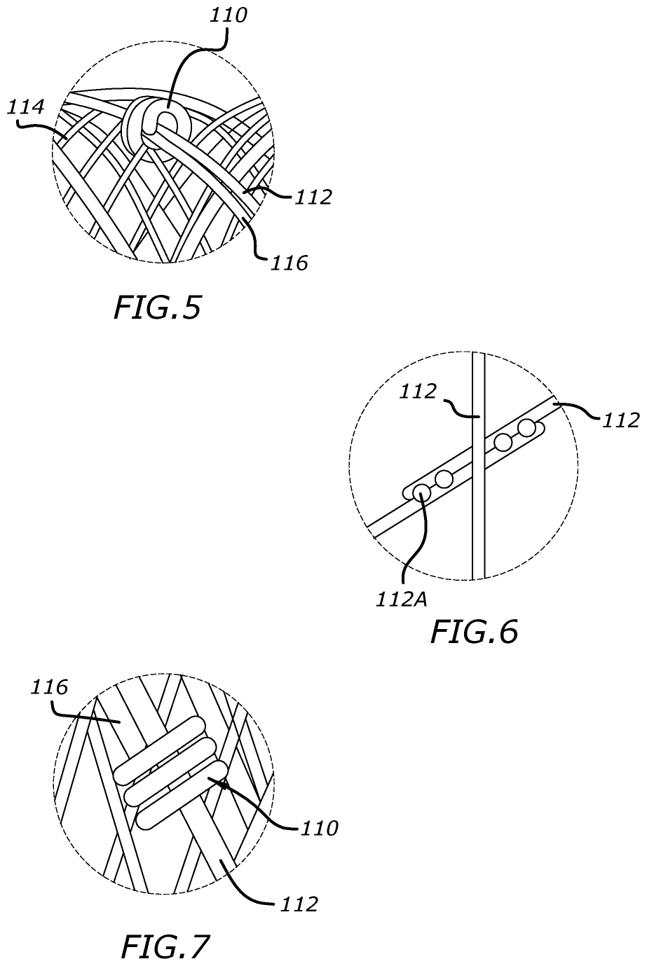

As seen in FIGS. 5 and 7, the ends of a connecting wire 116 may be connected or fixed to the outer wire 112 of the outer layer 102 (and/or optionally the inner wire 114 of the inner layer 104) to help prevent the connecting wire 116 from unwinding or coming apart from the two layers 102, 104. In one example, coiled wire ties 110 (e.g., a non-DFT wire 16 such as a tantalum or a non-super elastic alloy) may be used to connect the connecting wire 116 to the wire 112. In another example, each end of the connecting wire 116 may be wrapped around the wire 112. These coiled wire ties 110 can be connected in a manner that allows some movement of the wires 112 and 114 relative to each other or can be tightly connected in a manner that prevents movement of the wires 112 and 114 relative to each other. Coiled wire ties 110 can also optionally be used to connect both layers 102 and 104, separate of the connecting wire 116.

If a connecting wire 116 is not composed of a shape memory alloy such as Nitinol, it might instead be composed of a radiopaque material to enhance visualization. However, non-shape memory wires may be more difficult to configure to impart a desired amount of radially expansive force to achieve a relatively larger expanded stent size, particularly with one or more wires being composed of the flexible DFT wire 10. In especially tortuous or curved vessels, the two layers 102 and 104 may also exhibit force on each other and attempt to separate from each other, depending on many different factors. A pre-shape memory material/alloy such as Nitinol may act in a somewhat elastic manner when used for the connecting wire 116, returning to its original shape/configuration after the stress of deployment, whereas other materials without such super elastic properties may permanently change shape, depending on the magnitude and direction of the forces applied during delivery. In that manner, such a configuration with connecting wires composed of shape memory material may create a more resilient stent that is more resistant to damage.

Additionally, the shape memory material, such as Nitinol, of the connecting wire 116 may allow the connecting wire 116 to be heat set or pre-shaped during the manufacturing process. This pre-shaping can allow the connecting wire 116 to take on the shape of a helical coil with a predetermined diameter and pitch, similar to the wires of the inner layer 104 and outer layer 102 but, for example, with a different radial diameter so as to impart force on the other stent layers. Hence, pre-shaping the connecting wire 116 can allow for a different heat set diameter to the helical coil (or other shape) of the connecting wire 116 when in an expanded configuration versus the expanded configuration of the layers 102 and 104. In that respect, the layers 102 and 104 of the stent may be heat set after being braided, separately from the connecting wire 116, and the connecting wire 116 can be later connected and/or braided to the remaining layers 102, 104.

Hence, all three components, layers 102, 104, and the connecting wire 116, may radially expand and longitudinally contract in a similar manner, despite any sizing difference, exhibiting less resistance or force on each other. This can allow the stent 100 to open to a larger diameter (e.g., 5.0 mm or greater) than it otherwise would with connecting wire materials without super elastic properties (e.g., tantalum), and thereby provide better vessel wall apposition.

In that regard, the present embodiment of the stent 100 may specifically include an outer layer 102 composed of a single braided DFT wire 112 with a radiopaque inner core 12 and having a first configuration (e.g., braiding/winding pattern/angle, wire diameter), an inner layer 104 composed of one or a plurality of braided inner wires 114 having a second configuration (e.g., braiding/winding pattern/angle, wire diameter), and one or a plurality of pre-shaped connecting wires 116 (e.g., Nitinol) that connect the inner and outer layers 102, 104 together (e.g., a helical woven wire and/or a coiled wire).

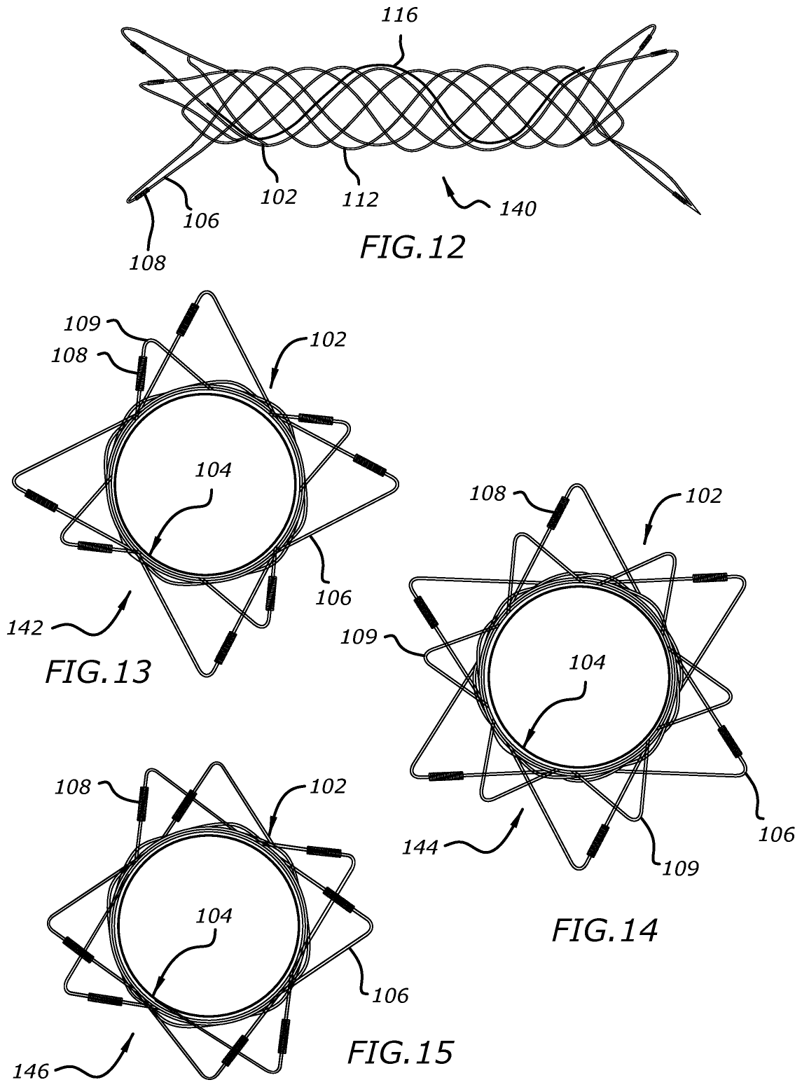

As previously discussed, the connecting wire 116 may be used with other stent embodiments having other layer configurations. For example, FIG. 12 illustrates a single layer stent 140 that is generally similar to the outer layer 102 of the previously discussed stent 100. The wires 112 may be heat set DFT wires 10 and therefore may have a relatively higher flexibility. One or a plurality of connecting wires 116 may be connected to and/or interwoven with the stent 140 in any of the previously discussed arrangements to provide the previously discussed performance advantages (e.g., expansion and anchoring).

In other examples, the stents 100′ and 100″ of FIGS. 8 and 9 may also include one or a plurality of connecting wires 116 similar to any of the previously discussed arrangements. The connecting wires 116 may be further braided and/or connected between only two layers or all of the layers. Additionally, different connecting wires 116 may be connected to different pairs of stent layers.

It should also be appreciated that different materials may be utilized for the connecting wire 116 other than Nitinol which was previously discussed. As further examples, the connecting wire 116 may be composed of DFT or tantalum wire. It has been shown, however, that Nitinol or DFT connecting wires 116 may provide better stent diameter recovery to keep the layers 102, 104 together when compared with tantalum connecting wires 116.

The present invention also includes a method of manufacturing a stent by pre-shaping or heat setting a shape to a connecting member 116 and then connecting and/or braiding/weaving the connecting member 116 to one or more stent layers.

One specific example method is described with regard to the dual layer stent 100 of FIGS. 2A-7, though it is applicable to any of the embodiment of this specification. In such a method, a shape memory connecting wire 116 (e.g., Nitinol) may be wrapped around a fixture or mandrel 130, as seen in FIG. 10. The connecting wire 116 may be wrapped to have a coil angle that matches that of one of the wire portions of the outer layer 102. The mandrel 130 may include guides, grooves, or similar physical features to help achieve a desired helical diameter and pitch. Optionally, the mandrel 130 may have a diameter that is larger than a mandrel that the remaining stent layers 102, 104 are braided on. The connecting wire 116 may then be heat set on the mandrel 130 to retain this coil shape and size. The connecting wire 116 can be further processed or finished as needed, such as via polishing, passivating, etching, or pickling.

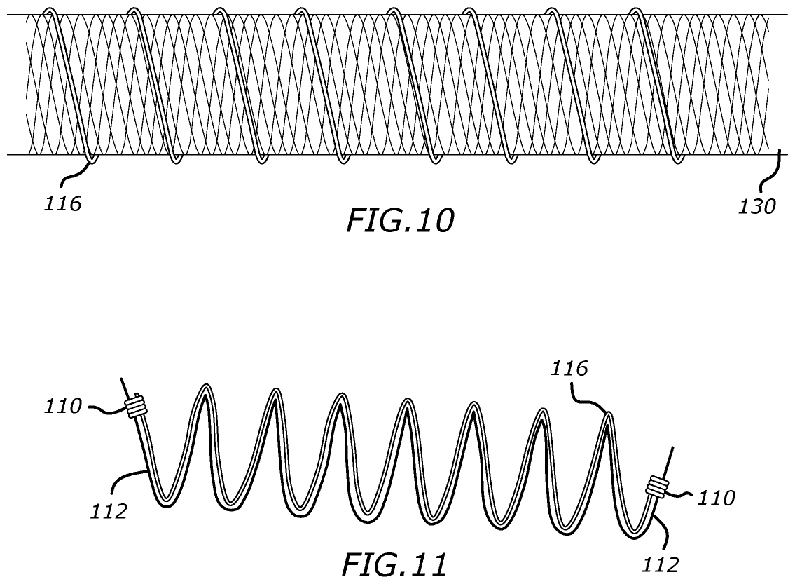

The woven outer layer 102 and inner layer 104 can then be brought together so that the inner layer 104 is positioned and aligned within the outer layer 202, or can be optionally braided on top of each other. These layers 102 and 104 may be heat set on a mandrel to set a predetermined radial size in an expanded configuration. Optionally, this mandrel diameter size may be smaller than that of the mandrel 130. The connecting wire 116 can then be woven through both layers 102 and 104 to generally following a similar path adjacent to one of the outer wires 112, but otherwise passing over and under both wires 112 and 114. The result is that the connecting wire 116 may have its helical, heat-set form that is interwoven with and generally matches with that of one or both of the other two layers 102, 104 (illustrated in FIG. 11). Alternately, the connecting wire may be braided in a helical direction that is rotationally opposite (opposing pitch) to that of the outer wires 112. Alternately, the connecting wire 116 may be located between the layers 102 and 104 without interweaving into the layers.

When the connecting wire 116 is in its desired position, wire ties or coils 110 (or other previously discussed connection mechanism) can be formed on each end of the connecting wire 216, as seen in FIG. 11. The wire ties 110 can be formed by wrapping a wire (e.g., tantalum) around both the connecting wire 116 and a portion of the outer wire 112. Alternately, the wire of the tie 110 can also be wrapped around the inner wire 114. Alternately, the ends of the connecting wire 116 may be wrapped around the outer wire 112 to form the wire ties 110, however, a non-shape memory material may provide greater resistance to deformation and thereby provide a stronger connection point. Additionally, wire ties 110 (or similar connections) may be included at other locations along the length of the connecting wire 116.

It should be appreciated that the pitch of the connecting wire 116 may vary along the length of the stent 100 as the connecting wire 116 is woven through the layers 102, 104 of the stent 100. As an example, a pitch of a first winding of the connecting wire 116 may be different from a pitch of a second winding of the connecting wire 116. Additionally, the direction of the winding may vary in different embodiments, with one example embodiment using right hand winding and another example embodiment using left hand winding. Further, the OD of the winding of the connecting wire 116 may vary in different embodiments.

The stent 100 of FIGS. 2A-7 includes five relatively larger loops 106 and five relatively smaller loops 109. However, additional numbers of loops and sizes of loops are also possible. In that regard, any of the stents described in this specification may include a plurality of larger loops 106 and smaller loops 109 that form an alternating pattern on one or more of its ends. For example, FIG. 13 illustrates a stent 142 with four pairs of larger and smaller alternating loops 109. In another example seen in FIG. 14, a stent 144 may include six pairs of larger and smaller loops 106, 109. Additionally, embodiments with only one size of end loop are also possible (e.g., all loops may be of a substantially uniform size). For example, FIG. 15 illustrates a stent 146 that includes eight pairs of similarly size loops 106. The embodiments of FIGS. 13-15 may be particularly suited for stents having a diameter greater than 5.00 mm, such as between about 6.0 mm to 8.0 mm, to improve stent opening and stability.

The long flares/loops 106 and short flares/loops 109 can each be oriented at about a 60-degree angle (relative to a horizontal plane extending through the axial/radial middle of the stent). The flare/loop sizes can vary based on the size of the stent as well. In various examples, the stent is sized from about 2.5-5 mm in diameter. In some embodiments, the stent may be sized greater than 5 mm in diameter, such as between 6-8 mm in diameter. This particular size may fit neurovascular arteries, which are smaller than arteries in the majority of the vasculature, and provide benefit as a scaffolding stent used to provide support against a neck region of an aneurysm for subsequent devices (e.g., embolic coils, or other occlusive agents) used to fill the aneurysm. Proper apposition of the stent may be particularly helpful in this target therapeutic regimen to ensure the stent does not migrate away from the aneurysm site, which could then allow embolic material to migrate when left without a supporting scaffold.

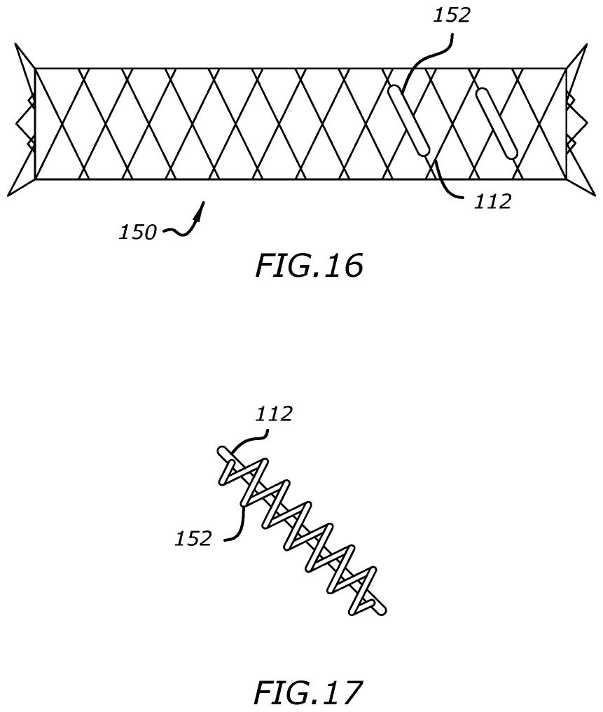

Any of the stent embodiments of this specification may include one or more reinforcing elements to help further increase the force with which a stent radially expands. For example, FIGS. 16 and 17 illustrate aspects of a stent 150 that is similar to stent 100 or 140. However, one or more regions of the stent 150 may include reinforcement elements 152 positioned over stent wire 112 (which may be a DFT wire 10) to introduce increased strength and stiffness along the one or more regions. It should be appreciated that the number, size, positioning, and orientation of any such reinforcement elements 152 may vary in different embodiments.

In typical braided stents, it can be difficult to fully expand the proximal end of the stent once the remaining portion of the stent is deployed. This can be particularly due to tortuous vasculature in which a stent is deployed. This problem may be magnified as stents are designed to be less stiff and more flexible, such as by using DFT wires 10 that are heat set. Therefore, introducing one or more reinforcement elements 152 along a portion of the stent 150, such as a proximal, distal, or medial region of the stent 150, may help augment opening force along this region, promoting ease of deployment. These reinforcement elements 152 can also be used in combination with the previously discussed connecting wire 116 so that both components provide radially expansive force on the stent 150.

The reinforcing element 152, in one example, may comprise a coil as is shown in greater detail in FIG. 17, where the reinforcing coil is wound around the DFT stent wire 112 of the stent 150. In other embodiments such as shown in FIG. 16, the reinforcing element 152 may comprise a tube that is placed over the DFT wire 152 along one or more regions of the stent. In one embodiment, the reinforcing element 152 may be attached to the wire 112 (e.g., via adhesive or welding) to fix the location. In another embodiment, the reinforcing element 154 may not be fixed and may be free to move (e.g., by sliding and/or rotating). In another embodiment, the reinforcing element 154 may be another linear wire element which is attached to a portion of the DFT wire 112 to “thicken” the associated DFT wire segment.

The reinforcing element 152, in one example, may be made of a strong shape memory material. A preferred example is Nitinol (e.g., either a Nitinol coil or a Nitinol tube), but other examples can include cobalt-chromium or stainless steel.

Where the reinforcing element 152 is a coil, as is shown in FIG. 17, this coil may have an associated stiffness or k-value associated with it. This stiffness/k-value may depend on a number of attributes including the material composition, the thickness of the coil, and how close wound the reinforcing coil is (i.e., the pitch). A higher k-value could be effected, for instance, by utilizing a relatively stiff material (e.g. radiopaque material such as gold, platinum, tungsten, palladium, tantalum, or non-radiopaque metals that are stiff), by using a closely-wound pitch for the coil, and/or by adjusting properties of the coil (e.g., the thickness of the wire comprising the coil, the overall width of the coil, and the overall length of the coiled reinforcing element 152).

The portion of the wire 112 which is underneath the reinforcing element 152 may have its own associated stiffness of k-value, as the wire forming the reinforcing element 152 may have its own corresponding “springiness” due to being wound in a helical, longitudinal manner along the stent 150. Note, this “springiness” will increase as the stent 150 is compressed and may help to propel the stent 150 open upon deployment. The k-value of the wire 112 will depend on the associated stiffness of DFT wire, the diameter of the wire, and the pitch of the wire comprising the DFT stent 150 (in other words, the helical/longitudinal wind pattern used to mechanically wind the stent 150).

The stent region shown in FIG. 17, where the reinforcing coil sits over a portion of the wire 112, can be thought of as two parallel springs and Hooke's law would yield a corresponding stiffness. Where the wire 112 has an associated stiffness k₁ and the reinforcing coil 152 has an associated stiffness k₂, the overall stiffness of this region will then be (k₁+k₂), in other words the combined stiffness will be higher. In this way, the reinforcing element 152 may serve to increase the associated stiffness at that region. This increased stiffness has certain advantages, for instance strengthening a particular region of the stent 150 to augment deployment force (helping the stent open) and promoting apposition against the vessel wall along the reinforced section.

Another advantage is that the augmented stiffness and the enhanced area that the reinforcing element takes up across the underlying wire will help adjacent cells of the stent 150 open. If adjacent cells cannot sufficiently open, these cells will contact the reinforcing element 152 (which has a higher surface area than the underlying and surrounding wire 112), and this contact force can help these other cells open.

The reinforcing element 152 can be placed in one or more regions along the DFT stent 150. For instance, it can be placed in roughly equidistant intervals (or alternatively, in random locations) over the length of the stent 150 to promote a consistent expansion and consistent enhanced stiffness across the entirety of the stent. Alternatively, it can be placed along solely the proximal section of the stent 150 (as shown in FIG. 16), in one or more locations along the proximal section in order to enhance strength and opening in the proximal region of the stent.

In an example embodiment, a pair of reinforcing elements 154 may be positioned at a location along the radial circumference of the stent 150. In such an embodiment, the reinforcing elements 152 may be aligned with each other along a longitudinal axis extending through the length of the stent 150. Additional reinforcing elements 152 may be positioned in such an embodiment at various other radial locations around the circumference of the stent 110, such as on the opposing side, as necessary to augment the stiffness of the stent 150.

In other example embodiments, the locations of the reinforcing elements 154 may vary from that shown in the figures. For example, in some embodiments, the reinforcing elements 152 may instead or additionally be positioned at or near the distal region of the stent 150. As a further example, the reinforcing elements 152 may instead be positioned on the opposing winds such that they are angled differently than is shown in the figures.

The reinforcing element 152 can be added in a variety of ways to the DFT wire 112 of the stent 150. The following techniques can be used regardless of whether the DFT stent 150 comprises solely one DFT wire, or a plurality of DFT wires. In one embodiment, the reinforcing element 152 may be slid over the respective wire segment before or during the winding procedure used to wind the stent 150.

In another embodiment, the wire can be cut near the region where the reinforcing element 152 is added to the wire, and once the reinforcing element 152 is appropriately placed, the wire may then be soldered or welded to the other cut section of the wire to reattach the two wire segments. One advantage of placing this wire attachment location near the reinforcing element 152 is that this will thicken the associated wire segment, which can help keep the reinforcing element 152 in a particular location and keep it from moving around.

In one example, the reinforcing element 152 is a Nitinol coil having an inner diameter of about 0.003 inches and an outer diameter of about 0.0065 inches. Where plural reinforcing elements 152 are used, they can be spaced in various ways, for instance one wire wind can separate two elements 152, more wire winds can separate the two elements, or the elements 152 can be spaced directly adjacent each other at adjacent winds.

While the embodiment of the stent 100 discussed primarily with regard to FIGS. 2A-7, as well as other locations, primarily discusses the use of a DFT wire 10 to make up the outer layer 102, alternately a wire composed entirely of radiopaque material can be used instead, such as gold, platinum, tungsten, platinum-tungsten, palladium, iridium, platinum-iridium, rhodium, tantalum, barium sulfate, bismuth subcarbonate, bismuth oxychloride, bismuth trioxide or combinations thereof. Hence, one aspect of the present invention includes a stent having a first braided layer braided from one or more radiopaque wires and a second braided layer braided from one or more shape memory wires, where the two layers are connected to each other.

In one embodiment, the present invention includes a stent comprising at least one layer braided from one or more wires; the at least one braided layer forming a stent body having a tubular shape and having a plurality of longer loops and a plurality of shorter loops disposed at its proximal end, its distal end, or both its proximal and distal ends; wherein the plurality of longer loops and the plurality of shorter loops form an overlapping and alternating pattern; and, a radiopaque marker positioned on at least one of the plurality of longer loops, adjacent to the stent body such that an adjacent shorter loop of the plurality of shorter loops does not contact or move across the radiopaque marker.

The term shape set is used within this specification to refer to an imparted secondary shape on a wire or similar component that is composed of a shape memory alloy such as Nitinol. Typically, such shape setting may occur by the application of heat when a component is placed in a desired shape that the component may return to after deformation within certain temperatures.

The term “about” may be used in this specification with regard to various numbers (e.g., dimensions). The use of this term should be understood to cover numbers within a range 5% above and 5% below a given number.

It should be understood that different aspects of the embodiments of this specification can be interchanged and combined with each other. In other words, additional embodiments are also specifically contemplated by combining different feature from different embodiments. Therefore, while specific embodiments are shown in the Figures, it is not intended that the invention necessarily be solely limited to those specific combinations.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

### Clauses

Exemplary embodiments are set out in the following numbered clauses.
Clause 1. A method of manufacturing a stent may comprise the steps of wrapping a shape memory connecting wire around a fixture; heat setting the shape of the shape memory connecting wire; and weaving the shape memory connecting wire through an outer stent layer and an inner stent layer located within the outer stent layer.
Clause 2. A method of forming a stent may comprise braiding a first stent layer and shape setting the first stent layer to have a secondary shape having an expanded tubular shape of a first diameter; shape setting a connecting wire to have an expanded shape of a second diameter that is the same size, larger, or smaller than the first diameter; and, connecting the connecting wire to at least the first stent layer.
Clause 3. A method of manufacturing a stent may comprise the steps of forming a first stent layer by braiding at least one first stent wire into a first tubular shape; forming a second stent layer by braiding one or more second stent wires into a second tubular shape; and connecting the first stent layer to the second stent layer.
Clause 4. A method according to any of the preceding clauses, wherein manufacturing a stent may further comprise the steps of forming a third stent layer by braiding one or more third stent wires into a third tubular shape and connecting the third stent layer between the first and second stent layers.
Clause 5. A method according to any of the preceding clauses, wherein a stent may further comprise the steps of connecting the first stent layer to the second stent layer and/or third stent layer by one or more connecting wires.
Clause 6. A method of delivering a stent may comprise the steps of positioning the stent within a delivery catheter in a radially compressed state; advancing the delivery catheter to a target location in a vessel; and releasing the stent from the delivery catheter within the vessel such that the stent expands into a radially expanded state, wherein step of releasing the stent from the delivery catheter may comprise actuating an implant detachment mechanism.
Clause 7. A method according to clause 6, wherein the stent comprises a first layer braided from DFT wire and a second layer braided from non-DFT wire.
Clause 8. A method according to clause 6, wherein the stent comprises at least one layer braided from DFT wire and a connecting wire comprises a shape-memory material that is pre-shaped to have an expanded shape.

What is claimed is:

1. A stent comprising:
an outer stent layer comprising at least one first stent wire braided into a first tubular shape; wherein the at least one first stent wire comprises a DFT wire; and,
an inner stent layer comprising one or more second stent wires braided into a second tubular shape;
wherein the outer stent layer is connected to the inner stent layer.

2. The stent of claim 1, wherein the second stent wires comprise DFT wires.

3. The stent of claim 1, wherein the second stent wires comprise non-DFT wires.

4. The stent of claim 1, further comprising a third stent layer comprising one or more third stent wires braided into a third tubular shape, and wherein the third stent layer is positioned between the outer stent layer and the inner stent layer, the outer stent layer is positioned within the inner stent layer, or the outer stent layer is positioned within the third stent layer.

5. The stent of claim 1, wherein the outer stent layer is braided with only a single DFT wire.

6. The stent of claim 5, wherein the inner stent layer is braided with a plurality of wires that are composed of a non-DFT material.

7. The stent of claim 1, further comprising one or more connecting wires connected to the outer stent layer and the inner stent layer; wherein the one or more connecting wires are composed of a shape memory material and have been shape set to have a secondary shape prior to connection with the outer stent layer and the inner stent layer.

8. The stent of claim 7, wherein the secondary shape of the one or more connecting wires has a diameter larger than an expanded diameter of the stent.

9. The stent of claim 7, wherein the secondary shape of the one or more connecting wires is helical.

10. The stent of claim 9, wherein a pitch of the helical secondary shape of the one or more connecting wires is substantially similar to a braid pitch of the at least one first stent wire.

11. The stent of claim 9, further comprising a first coil disposed around a first end portion of one of the one or more connecting wires and around a first portion of the at least one stent wire, and a second coil disposed around a second end portion of one or the one or more connecting wires and around a second portion of the at least one stent wire.

12. The stent of claim 7, wherein the one or more connecting wires comprise a first connecting wire connected along a first region of the stent and a second connecting wire connected along a second region of the stent.

13. A stent comprising:
a first stent layer comprising at least one first stent wire braided into a first tubular shape; wherein the at least one first stent wire comprises a DFT wire; and,
one or more connecting wires connected to the first stent layer;
wherein the one or more connecting wires are composed of a shape memory material and have been shape set to have a secondary shape prior to connection with the first stent layer.

14. The stent of claim 13, further comprising a second stent layer braided from a plurality of second stent wires; wherein the one or more connecting wires are interwoven with the first stent layer and the second stent layer.

15. The stent of claim 13, wherein the secondary shape of the one or more connecting wires has a diameter larger than an expanded diameter of the stent.

16. The stent of claim 13, wherein the secondary shape of the one or more connecting wires is helical.

17. The stent of claim 13, further comprising a first coil disposed around a first end portion of one of the one or more connecting wires and around a first portion of the at least one stent wire, and a second coil disposed around a second end portion of one or the one or more connecting wires and around a second portion of the at least one stent wire.

18. The stent of claim 13, wherein the one or more connecting wires comprise a first connecting wire and a second connecting wire, and wherein the first connecting wire and the second connecting wire are connected to at least the first stent layer such that they partially overlap, fully overlap, or are adjacent to each other along a length of the stent.

19. The stent of claim 13, wherein the first stent layer has a first shape set expanded size and the one or more connecting wires have a second shape set expanded size that is larger than the first shape set expanded size.

20. A stent comprising:
a first stent layer means for forming a braided layer of a stent comprising at least one first stent wire braided into a first tubular shape; wherein the at least one first stent wire comprises a DFT wire means for being visualized by radiography; and,
one or more connecting wire means for connecting to the first stent layer means;
wherein the one or more connecting wire means are composed of a shape memory material and have been shape set to have a secondary shape prior to connection with the first stent layer means.
